(19) United States
(12) Patent Application Publication
(10) Pub. No.: US 2005/0256495 A1
(43) Pub. Date: Nov. 17, 2005

(54) Composite web and use in an absorbent article

(75) Inventors: Daniel Robert Schlinz, Greenville, WI (US); Barbara Ann Gossen, Oshkosh, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/836,490

(22) Filed: Apr. 29, 2004

(51) Int. Cl. A61F 13/15; A61F 13/20

(52) U.S. Cl. 604/385.201; 604/385.01; 604/385.02

(57) ABSTRACT

A composite web has a first fastening material in a first longitudinally extending zone on a first outer surface, a second fastening material in a second longitudinally extending zone on the first outer surface, and at least a portion of a third fastening material on a longitudinal centerline on a second outer surface. A disposable absorbent article has a dual fastening system comprising a pair of front ear portions and a pair of back ear portions formed from a composite web. The composite web has first, second, and third fastening materials. Each back ear portion has a fastening material configured to engage the outer cover and each front ear portion has a fastening material configured to engage the body facing surface of the liner.

### BACKGROUND OF THE INVENTION

Disposable diapers, and other absorbent articles, are typically manufactured from a high-speed moving web of nonwoven or other materials. Layers and components are introduced and brought together at various points to the moving web and, when all of the desired components are present, the web is cut into individual articles. As three-dimensional articles, absorbent articles are generally thin for comfort and fit purposes. Therefore, the primary dimensions of such articles are their length (longitudinal direction) and width (lateral direction). The thickness of an absorbent article is composed of piles of several of the article components that are generally coterminous with each other and form the “chassis” of the absorbent article. These components may include the outer cover (also known as the backsheet), the absorbent core, and the bodyside liner (also known as the topsheet). Other components are often attached to the chassis to form the complete article. These separate components include containment flaps, leg elastics, waist elastics and fastening system elements. As can be generally appreciated, the complexity and difficulty of the absorbent article manufacturing process increases as the number of separate components that must be introduced to the moving web increases. An associated challenge is the placement of each of those components in the correct location on the high-speed moving web.

The chassis of an absorbent article is generally understood to include a front waist portion, a crotch portion and a back waist portion.. Fastening systems have been developed for joining the waist portions together around the waist of the wearer of the article. In order to provide coverage of the article over the hips of the wearer and to improve placement of the fasteners, absorbent articles have evolved to include “ear” portions. The ear portions of a diaper can serve many functions; for example, the ear portions can aid placement of the waist portions prior to fastening and the ear portions can provide coverage of the article around the hips of the wearer of the article. With some absorbent articles, the ear portions can form at least a part of the waist opening and the leg openings.

The ear portions may be attached to the front waist portion, the back waist portion or there may be two sets; one in the front and one in the back. The ear portions generally extend laterally (perpendicularly) away from the chassis of the absorbent article. The ear portions can be made of a variety of materials and with disposable absorbent articles, they are typically made, at least partially, from a nonwoven material. At present, commercially available premium diapers tend to have back ear portions that are constructed from an elastic nonwoven material, such as a neck-bonded laminate. However, ear portions can be constructed of non-elastic or simply extensible nonwoven materials too and sometimes it is even desirable to have the ear portion constructed of a non-elastic nonwoven or for the ear portion to have a non-elastic/non-extensible portion attached to an elastic material. Therefore, in some cases, the ear portion can be constructed of more than one type of material. The addition of a non-elastic portion at the distal (furthest away from the chassis) end of the ear portion can simplify the application of the absorbent article because it gives the caregiver or wearer something sturdy to grab onto as they bring the waist portions of the article together for fastening.

While ear portions increase the ease with which absorbent articles can be applied and while they improve the fit of the articles, there are limitations associated with their attachment to the chassis during manufacture. The limitations generally relate to the ear portions not being integral with the chassis and therefore, requiring separate placement onto the high speed moving web of materials. Typically, it is desirable to place the ear portions near the edges of the front and back waist portions. Consequently, it is necessary to “register” the ear portions to a desired location in relation to the front and back waist edges of individual absorbent articles. In general, the closer the ear portions are to the waist edges, the better they perform their function. For example, if a diaper article includes a stretchable back waistband, registration of a stretchable back ear portion with the back waist edge results in better tensioning/performance of the stretchable waistband when the diaper is applied to a wearer. However, current manufacturing techniques limit precise and consistent registration of the ear portions relative to the waist edges of the article. The process of applying ear portions to the web is further complicated when the finished article is intended to include ears in both the front and the back waist regions. Commercially-available diapers having both front and back ear portions have typically had front and back ears that are made from different types of nonwoven materials. Therefore, there are two separate streams of ear portion materials that have to be introduced to the main web, further complicating manufacturing.

The manufacturing and registration of the ear portions are yet further complicated with the addition of dual (or secondary) fasteners. Dual fasteners may include primary fastening means and secondary fastening means. The primary fastening means maintain the waist portion of the diaper in an overlapping configuration while the secondary fastening means typically are positioned to reduce shifting of the overlapping portions. Typically, the addition of the secondary fastening means involves the addition of yet another separate stream of ear portion material to the main web and the addition of a cut and place module or similar equipment to sever the secondary fastener and place it onto the absorbent article. Therefore, there exists a need to simplify the addition and registration of secondary fasteners to absorbent article manufacturing processes.

### SUMMARY OF THE INVENTION

In response to the difficulties and problems discussed above, new composite webs have been invented. Additionally, new disposable absorbent articles, that include secondary fasteners made from the new composite webs, have been invented.

One aspect relates to a composite web. The composite web has a base web and the base web has opposed first and second outer surfaces, a longitudinal centerline, and opposed first and second longitudinal side edges. The base web also has a first longitudinally extending zone lying between the longitudinal centerline and the first longitudinal side edge and a second longitudinally extending zone lying between the longitudinal centerline and the second longitudinal side edge. The composite web has first, second, and third fastening materials. The first fastening material is situated in the first longitudinally extending zone on the first outer surface of the base web. The second fastening material is situated in the second longitudinally extending zone on the first outer surface of the base web. The third fastening material is situated at least partially on the longitudinal centerline on the second outer surface of the base web.

In various embodiments of the composite web, the first and/ or second and/or third fastening material may be a discontinuous strip of material (i.e., discrete pieces). In embodiments wherein the first and second fastening materials are discontinuous strips of material, the first fastening material and the second fastening material may be configured in offset relation with one another.

In various embodiments of the composite web, at least a portion of the first and/or second fastening material may be situated adjacent a longitudinal side edge of the base web.

In various embodiments of the composite web, the first fastening material may be the same as the second fastening material.

Another aspect relates to a disposable absorbent article. The disposable absorbent article has opposed side edges, a front waist region, a back waist region, a crotch region extending between and connecting the waist regions, a longitudinal direction and a lateral direction. The disposable absorbent article also has an outer cover having a body facing surface and a garment facing surface; a liner superposed over the body facing surface of the outer cover, the liner having a body facing surface and a garment facing surface; an absorbent core disposed between the garment facing surface of the liner and the body facing surface of the outer cover; and a dual fastening system. The dual fastening system has a pair of front ear portions and a pair of back ear portions.

The pair of front ear portions and the pair of back ear portions are formed from a composite web. The composite web has a base web having opposed first and second outer surfaces, a longitudinal centerline, opposed first and second longitudinal side edges, a first longitudinally extending zone lying between the longitudinal centerline and the first longitudinal side edge, and a second longitudinally extending zone lying between the longitudinal centerline and the second longitudinal side edge. The composite web also has first, second and third fastening materials. The first fastening material is situated in the first longitudinally extending zone on the first outer surface of the base web. The second fastening material is situated in the second longitudinally extending zone on the first outer surface of the base web. At least a portion of the third fastening material is situated on the longitudinal centerline on the second outer surface of the base web.

Each back ear portion extends laterally outward from a side edge in the back waist region. Each back ear portion has a garment facing first outer surface, a body facing second outer surface and the third fastening material. The third fastening material is situated on at least a portion of the second outer surface of each back ear portion. The third fastening material is configured to engage at least a portion of the garment facing surface of the outer cover.

Each front ear portion extends laterally outward from a side edge in the front waist region and has a garment facing first outer surface, a body facing second outer surface and one of the first fastening material or the second fastening material. The first or second fastening material is situated on at least a portion of the first outer surface of each front ear portion. The first and second fastening materials are configured to engage at least a portion of the body facing surface of the liner.

In various embodiments of the disposable absorbent article, the first and/or second fastening material may be a discontinuous strip of material (i.e., discrete pieces). In some embodiments, wherein both the first fastening material and the second fastening material are discontinuous strips of material, the first fastening material and the second fastening material may be configured in offset relation with one another when part of the composite web. In some embodiments, the third fastening material may be a discontinuous strip of material (i.e., discrete pieces). In some embodiments, wherein the first fastening material comprises discrete pieces, the discrete pieces of the first fastening material may be entirely located within the first longitudinally extending zone of the composite web. In some embodiments, wherein the second fastening material comprises discrete pieces, the discrete pieces of the second fastening material may be entirely located within the second longitudinally extending zone of the composite web.

In various embodiments of the disposable absorbent article, the first fastening material may be similar or identical to the second fastening material.

In various embodiment of the disposable absorbent article, the first fastening material, the second fastening material, and the third fastening material may be hook type fastening materials and the outer cover may be configured to function as a complementary loop type fastener and the liner may be configured to function as a secondary loop type fastener. In various embodiments, the disposable absorbent article may further have at least one attachment panel located on the garment facing surface of the outer cover to which the third fastening material may be releasably engaged.

Another aspect relates to a method of making a disposable absorbent article that defines a front waist region, a back waist region, a crotch region that extends between and connects the waist regions, a longitudinal direction and a lateral direction. The method includes providing a first continuous web. The first continuous web has a base web. The base web has opposed first and second outer surfaces, opposed first and second longitudinal side edges, a longitudinal centerline, a first longitudinally extending zone lying between the longitudinal centerline and the first longitudinal side edge, and a second longitudinally extending zone lying between the longitudinal centerline and the second longitudinal side edge. The first continuous web also has first, second, and third fastening materials. The first fastening material is situated in the first longitudinally extending zone on the first outer surface of the base web. The second fastening material is situated in the second longitudinally extending zone on the first outer surface of the base web. At least a portion of the third fastening material is situated on the longitudinal centerline on the second outer surface of the base web.

The method further includes selectively cutting the first continuous web to form two ear portion webs wherein each ear portion web includes a repeating pattern of a back ear portion and front ear portion. The back ear portion includes the third fastening material and the front ear portion includes one of the first fastening material or the second fastening material. The method further includes cutting and orienting the two ear portion webs to form pairs where each pair includes one of the back ear portions and one of the front ear portions. In various embodiments, the cutting and orienting step may be accomplished by crossing the two ear portion webs and selectively cutting the two ear portion webs to form pairs where each pair includes a back ear portion and a front ear portion. In other embodiments, the cutting and orienting step may be accomplished by cutting the two ear portion webs to form pairs where each pair includes a back ear portion and a front ear portion and rotating each pair 180 degrees.

The method further includes providing a second continuous web of interconnected absorbent chassis wherein each of the absorbent chassis has an exterior surface, an interior surface opposite the exterior surface, a pair of laterally opposed side edges and a pair of longitudinally opposed waist edges.

The method further includes attaching the pairs of ear portions on both of the laterally opposed side edges of the interconnected absorbent chassis wherein the pairs of ear portions are attached in alignment with the waist edges of adjacent absorbent chassis and selectively cutting the second continuous web of interconnected absorbent chassis into discrete absorbent articles wherein each discrete absorbent article includes two laterally opposed front ear portions and two laterally opposed back ear portions. The two laterally opposed front ear portions have one of the first or second fastener material and the two laterally opposed back ear portions have the third fastener material.

### BRIEF DESCRIPTION OF THE DRAWINGS

The present invention will be more fully understood and further advantages will become apparent when reference is made to the following detailed description of the invention and the accompanying drawings wherein like numerals represent like elements. The drawings are merely representative and are not intended to limit the scope of the appended claims.

FIG. 1A representatively shows one embodiment of an exemplary composite web;

FIG. 1B representatively shows a method by which the articles of the invention may be made;

FIG. 1C representatively shows an alternative method by which the articles of the invention may be made;

FIG. 2 representatively shows a pattern into which a composite material web may be cut in such a way that the back ear portions and front ear portions are nested together;

FIG. 3 representatively shows two individual absorbent articles adjacent each other as part of a continuous or unitary nonwoven web;

FIG. 4 representatively shows a composite material web from which nested front and back ear portions can be formed;

FIG. 5 representatively shows an alternative composite material web from which nested front and back ear portions can be formed;

FIG. 6 representatively shows another alternative composite material web from which nested front and back ear portions can be formed;

FIG. 7 representatively shows a plan view of the body facing surface of a disposable absorbent article of the invention;

FIG. 8 representatively shows a further alternative composite material web from which nested front and back ear portions can be formed; and

FIG. 9 representatively shows a web of material from which front and back side ear portions can be formed for use on a prefastened absorbent article, such as a diaper pant.

### DETAILED DESCRIPTION OF THE INVENTION

The present disclosure of the invention will be expressed in terms of its various components, elements, constructions, configurations, arrangements and other features that may also be individually or collectively referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

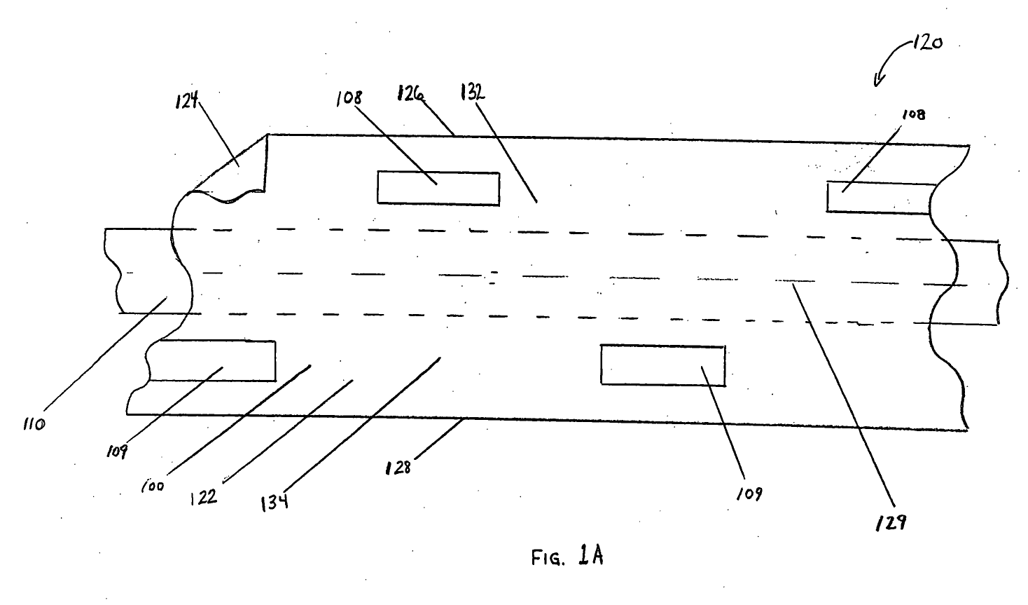

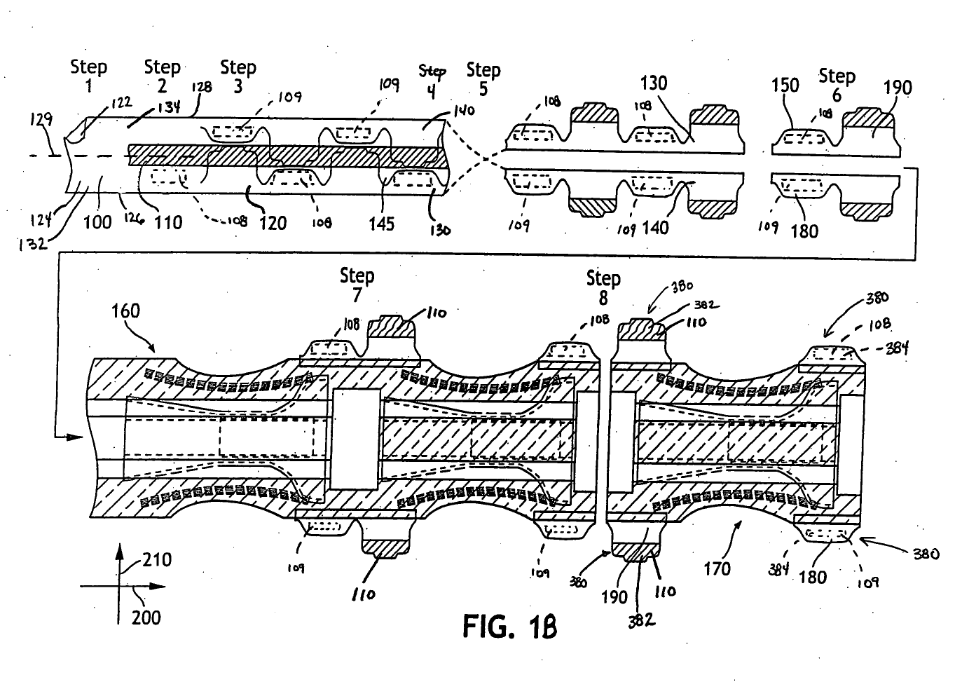

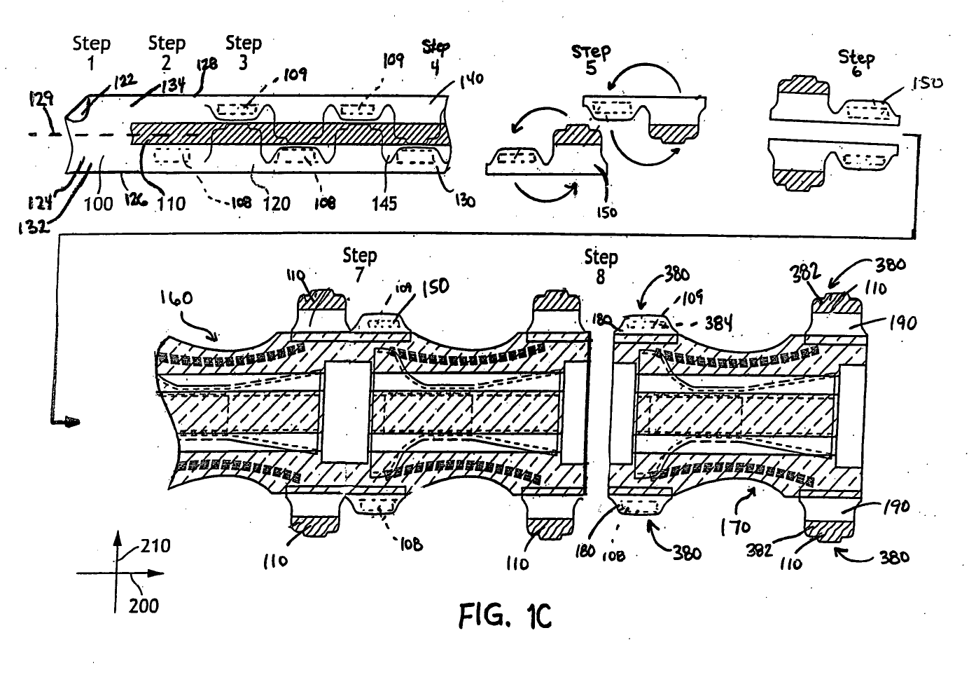

In a first aspect, the present invention relates to a composite web, a portion of which is representatively illustrated at 120 in FIG. 1A. The composite web 120 has a base web 100 having a first outer surface 122 and second outer surface 124, opposed first longitudinal side edge 126 and second longitudinal side edge 128, and a longitudinal centerline 129 (representatively illustrated with a dashed line in FIG. 1A). The base web 100 has a first longitudinally extending zone 132 lying between the longitudinal centerline 129 and the first longitudinal side edge 126. The base web 100 has a second longitudinally extending zone 134 lying between the longitudinal centerline 129 and the second longitudinal side edge 128. The base web 100 has at least one first fastening material 108 situated in the first longitudinally extending zone 132 on the first outer surface 122. In some embodiments, the first fastening material 108 is located entirely within the first longitudinally extending zone 132. The base web 100 has at least one second fastening material 109 situated in the second longitudinally extending zone 134 on the first outer surface 122. In some embodiments, the second fastening material 108 is located entirely within the second longitudinally extending zone 134. The base web 100 has a third fastening material 110 situated on the second outer surface 124. At least a portion of the third fastening material 110 is situated on the longitudinal centerline 129. In various embodiments, the base web 100 may comprise an integral web, as illustrated in FIG. 1A, or a plurality of connected sections.

In various embodiments, the first fastening material 108 may be a continuous strip of material or the first fastening material 108 may be more than one discontinuous strip of material (i.e., discrete pieces), as illustrated in FIG. 1A. Similarly, the second fastening material 109 may be a continuous strip of material or the second fastening material 109 may be more than one discontinuous strip of material (i.e., discrete pieces), as illustrated in FIG. 1A. In embodiments wherein the first and second fastening materials 108 and 109 are discontinuous strips of material, the strips may be configured in offset relation with one another as illustrated in FIG. 1A. Alternatively, the strips of the first fastening material 108 and the second fastening material 109 may be configured to at least partially align with one another. As used herein, the term “offset” means to situate two or more discontinuous strips of material such that an imaginary line drawn perpendicularly to the longitudinal centerline 129 and through any portion of one discontinuous first fastening material 108 does not pass through any portion of one discontinuous second fastening material 109. As used herein, the term “align” means to situate two or more discontinuous strips of material such that an imaginary line drawn perpendicularly to the longitudinal centerline 129 and through any portion of one discontinuous first fastening material 108 would also pass through a portion of one discontinuous second fastening material 109.

In some embodiments the third fastening material 110 may be a continuous strip of material as illustrated in FIG. 1A. Alternatively, the third fastening material 110 may be a discontinuous strip of material comprising two or more discrete third fastening material pieces 110.

The first fastening material 108 and/or the second fastening material 109 may be situated away from the respective longitudinal side edges 126 and/or 128 as illustrated in FIG. 1A. Alternatively, at least a portion of the first fastening material 108 and/or the second fastening material 109 may be situated adjacent the respective longitudinal side edges 126 and/or 128. In various embodiments, the first fastening material 108 may be similar to the second fastening material 109. For example, both the first fastening material 108 and the second fastening material 109 may be hook type fasteners. The first and second fastening material 108 and 109 need not be identical, but may, in some embodiments, be made of the same material.

In a second aspect, the present invention relates to disposable absorbent articles that have attached front ear portions, back ear portions, primary fasteners, and secondary fasteners that are formed from composite webs similar to the composite webs described above, for example, composite web 120 of FIG. 1A, and the composite webs described hereafter. The composite web can include one or more nonwoven or fastening materials. Typically, disposable absorbent articles include a front waist region, a back waist region and a crotch region that extends between and connects the waist regions. Absorbent articles also typically define a longitudinal direction and a lateral direction. Absorbent articles include an absorbent chassis that defines an exterior surface (or garment facing surface), an interior surface (or body facing surface) opposite the exterior surface, a pair of laterally opposed side edges and a pair of longitudinally opposed waist edges. As used herein, the term “disposable” refers to articles which are intended to be discarded after a limited use and that are not intended to be laundered or otherwise restored for reuse.

The present invention can also be used to address problems associated with the complexity of attaching front ear portions and back ear portions to the absorbent chassis of a disposable absorbent article, particularly when the front ear portions and the back ear portions are made of different materials. The articles of the invention can have front ear portions and back ear portions that are in improved registration with the absorbent chassis. Further, the process of the invention can reduce waste of materials because the front ear portions and the back ear portions are formed from a single web of material as opposed to two or more material webs. Further yet, articles of the invention can also be used to address problems associated with the complexity of attaching secondary fasteners to disposable absorbent articles.

The disposable absorbent articles of the present invention will be described in terms of a disposable diaper article that is adapted to be worn by infants about the lower torso. In particular, the disposable absorbent articles will be described in terms of a disposable diaper having a pair of opposed back ear portions and a pair of opposed front ear portions and a dual fastening system. It is understood that the articles of the present invention are equally adaptable for other types of absorbent articles such as adult incontinent products, training pants, feminine hygiene products and other personal care or health care garments.

FIG. 1B representatively illustrates a method by which the disposable diapers can be made. Step 1 of the method includes providing a base web 100 of material to be used for forming the back ear portions and the front ear portions. The base web 100 having a first outer surface 122 and second outer surface 124, opposed first longitudinal side edge 126 and second longitudinal side edge 128, and a longitudinal centerline 129 (representatively illustrated with a dashed line in FIG. 1B). The base web 100 has a first longitudinally extending zone 132 lying between the longitudinal centerline 129 and the first longitudinal side edge 126. The base web 100 has a second longitudinally extending zone 134 lying between the longitudinal centerline 129 and the second longitudinal side edge 128. The base web 100 may be formed of an extensible or an elastic material, such as a necked bonded laminate (hereinafter “NBL”). Alternatively, the base web 100 may be formed of a combination of a generally non-extensible material and an extensible material such as a combination of an SMS and an NBL. An example of a suitable material for base web 100 is a 1.5 ounce per square yard (hereinafter “osy”) spunbond/meltblown/spunbond (hereinafter “SMS”) nonwoven material. Additional suitable materials for the base web 100 include “bonded carded webs” (“BCW”); heavy-weight spunbond; and film-based materials such as film-based or film-like based laminates.

Typically, it is desirable to provide back ear portions, front ear portions, or both with a fastening material so that one or the other or both of the ear portions is able to secure the back waist and front waist regions together. For such product configurations, a web of a first fastening material 108, a web of a second fastening material 109, and a web of a third fastening material 110 may be fed in association with and laminated to the base web 100 to form a composite material web 120. In various embodiments, the first fastening material 108, the second fastening material 109, and/or the third fastening material 110 may be continuous or discontinuous (i.e., discrete pieces). The embodiment illustrated in FIG. 1B shows a discontinuous first fastening material 108, a discontinuous second fastening material 109, and a continuous third fastening material 110. The first fastening material 108 is in the first longitudinally extending zone 132 on the first outer surface 122. In various embodiments, the first fastening material 108 may be situated entirely within the first longitudinally extending zone 132. The second fastening material 109 is situated in the second longitudinally extending zone 134 on the first outer surface 122. In various embodiments, the second fastening material 109 may be situated entirely within the second longitudinally extending zone 134. The third fastening material 110 is situated on the second outer surface 124. At least a portion of the third fastening material 110 is situated on the longitudinal centerline 129. Lamination of first, second, and third fastening materials 108, 109, and 110 to the base web 100 is illustrated as Steps 2 and 3 in FIG. 1B.

A next step for forming articles according to one embodiment of the invention is to die cut the composite material web 120 into a right continuous composite web 130 and a left continuous composite web 140 in such a way that the back ear portions and the front ear portions being formed are nested with each other. Die cutting of the composite web 120 is illustrated in FIG. 1B as Step 3. The web material located between and not part of the back and front ear portions is referred to as trim waste 145. While not shown in FIG. 1B, the trim waste is removed from the system as Step 4. Next, shown as Step 5 in FIG. 1B, right continuous composite web 130 and left continuous composite web 140 are transposed, that is the webs 130 and 140 are crossed so that they end up oriented 180 degrees opposite of their initial orientation. The right continuous composite web 130 and left continuous composite web 140 each have a sequence of back ear portions 190 and front ear portions 180. The sequence depicted in FIG. 1B of alternating back and front ear portions may be used with absorbent articles that are formed “back to front” (meaning the back or rear portion of one article is adjacent to the front portion of the next article). If the absorbent articles are being formed “front to front”/“back to back”, then the sequence of ear portions on the right and left continuous composite webs 130, 140 would be front, front, back, back etc. Step 6 of FIG. 1B depicts the right continuous composite web 130 and the left continuous composite web 140 being cut into discrete front and back ear segments (or pairs) 150. In Step 7, the front and back ear segments/pairs 150 are attached to a continuous absorbent article web 160. The absorbent article web 160 has a machine direction 200 and a cross-direction 210. Finally, Step 8 of FIG. 1B depicts the continuous absorbent article web 160 being cut into individual absorbent articles 170. Each absorbent article 170 includes its own pair of opposed front ear portions 180 with first and second fastening material 108 and 109 and pair of opposed back ear portions 190 with third fastening material 110. A benefit of the front and back ear portions 180, 190 being made from the same composite web 120 is that the ear portions can be applied to the article web 160 at the same time. Further, the front and back ear portions 180, 190 can be in improved registration with the waist edges of the resulting absorbent articles 170. Additionally, first fastening material 108, second fastening material 109, and third fastening material 110 can be in improved registration with the absorbent article 170. The first fastening material 108, the second fastening material 109, and the third fastening material 110 can function together as a dual fastening system 380. The dual fastening system 380 includes primary fasteners 382 and secondary fasteners 384. The third fastening material 110 may be utilized as a primary fastener 382 and the first and second fastening materials 108 and 109 together may be utilized as a secondary fastener 384. In various configurations, the first and second fastening materials 108 and 109 may comprise the same fastening material or different fastening materials.

An alternative to the method illustrated in FIG. 1B is illustrated in FIG. 1C. In FIG. 1C, the Steps 1, 2, 3, and 4 are the same as discussed above. In Step 5, the right continuous composite web 130 and left continuous composite web 140 are cut into discrete front and back ear segments (or pairs) 150 and rotated. That is, pairs 150 are rotated 180 degrees so that they end up oriented as illustrated in Step 6 of FIG. 1C. In Step 7, the front and back ear segments/pairs 150 are attached to a continuous absorbent article web 160. The absorbent article web 160 has a machine direction 200 and a cross-direction 210. Finally, Step 8 of FIG. 1B depicts the continuous absorbent article web 160 being cut into individual absorbent articles 170. Each absorbent article 170 includes its own pair of opposed front ear portions 180 with first and second fastening material 108 and 109 and pair of opposed back ear portions 190 with third fastening material 110.

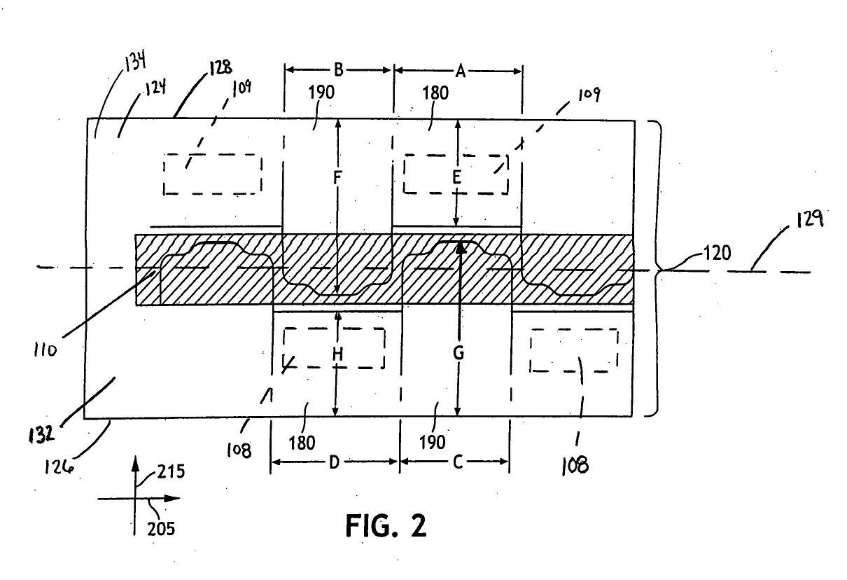

FIG. 2 representatively depicts a pattern into which a composite web 120 may be cut such that the front and back ear portions 180, 190 are nested together. The ear portions have a length that is measured in direction 215 and a width that is measured in direction 205. When the ear portions are attached to the absorbent article web 160, the length of the ear portions is aligned with (parallel to) the cross-direction 210 of the absorbent article web 160 and the width of the ear portions is aligned with (parallel to) the machine direction 200 of the absorbent article web 160. Two of the back ear portions 190 depicted in FIG. 2 have lengths “F” and “G” and widths “B” and “C”. Two of the front ear portions 180 depicted in FIG. 2 have lengths “E” and “H” and widths “A” and “D”. As depicted in FIG. 2, the lengths “F” and “G” of the back ear portions 190 are greater than the lengths “E” and “H” of the front ear portions 180 that the back ear portions 190 are nested with. More specifically, the sum of lengths “F” and “G” is greater than the sum of lengths “E” and “H”. Additionally, the width “A” of one front ear portion 180 is approximately equal to the width “D” of the diagonally opposite front ear portion 180. Likewise, the width “B” of one back ear portion 190 is approximately equal to the width “C” of the diagonally opposite back ear portion 190. In another aspect, the relative dimensions of the front and back ear portions 180, 190, the sum of the widths “A” and “B” of adjacent back and front ear portions 180, 190 is approximately equal to the sum of the widths “C” and “D” of the opposing, adjacent back and front ear portions 180, 190.

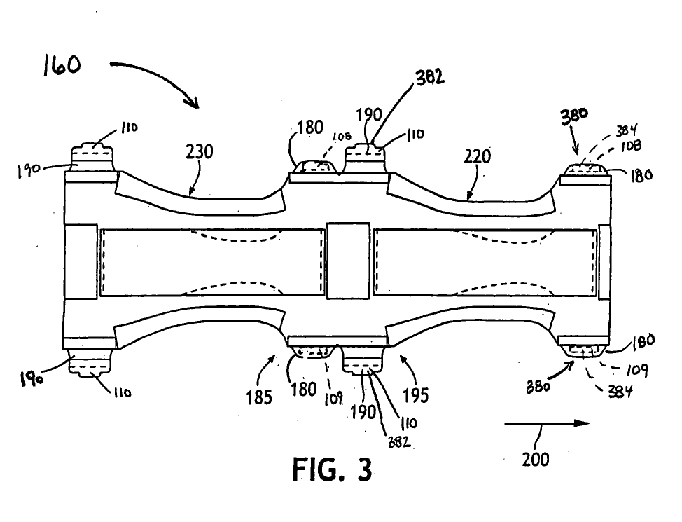

As previously described in relation to Step 8 of FIG. 1B, the continuous absorbent article web 160 is cut into individual absorbent articles 170 at the “final cut off” point in the process. FIG. 3 depicts two adjacent articles from a representative continuous absorbent article web 160 immediately before the final cut off. At the final cut off, the front ear portions 180 will be separated from the back ear portions 190 that they were originally continuous with (based on formation from the composite web 120). The articles depicted in FIG. 3 are being formed in a “back to front” configuration; that is, the back waist region 195 of one article is adjacent the front waist region 185 of the next article. The machine direction 200 of the absorbent article web 160 is depicted in FIG. 3 in order to distinguish between the first article 220 formed and the second article 230 formed. Therefore, the back ear portions 190 attached to the first article 220 are adjacent the front ear portions 180 attached to the second article 230 and the adjacent ear portions are separated from each other when the first article 220 and the second article 230 are separated. When the first article 220 and the second article 230 are separated from each other at the final cut off, the cut off forms the waist or longitudinal edges of the adjacent articles. Use of this technique of the invention for applying ear portions can result in the ear portions being in near perfect registration with the waist edges of the articles. That is, the attachment edge of the ear portion comes right to the waist edge instead of being somewhat over or below the waist edge. Additionally, the placement of the first, second and third fastening materials 108, 109, and 110 are in near perfect registration with the absorbent articles and one another. In a further processing step not depicted in FIG. 3, it is possible for front ear portions 180 of the articles to be attached to the back ear portions 190 of the articles to form prefastened disposable absorbent articles. The ear portions may be attached using techniques known to those of skill in the art including ultrasonic bonding, adhesives and mechanical fastening materials.

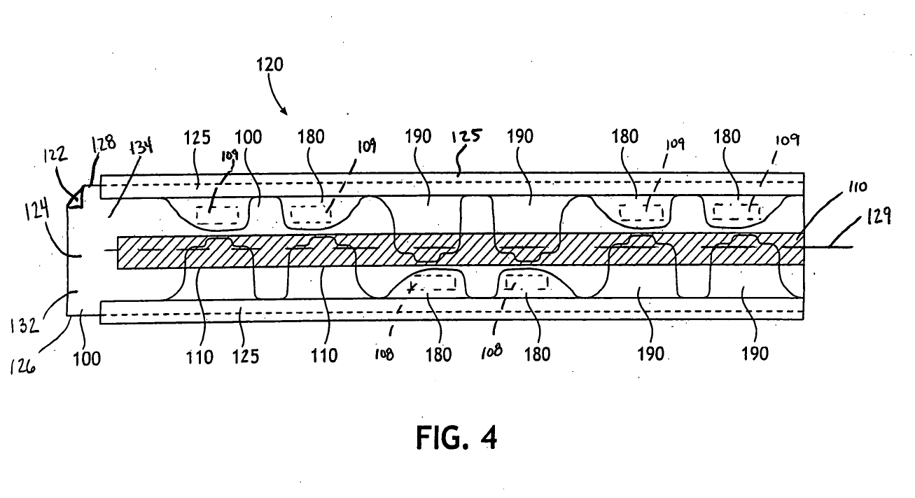

FIG. 4 representatively illustrates a composite web 120 from which nested front and back ear portions can be formed. The composite web 120 depicted in FIG. 4 representatively illustrates a nesting pattern for the front ear portions 180 and the back ear portions 190 of front ear portion 180; front ear portion 180; back ear portion 190; and back ear portion 190. When the composite web 120 is split into complementary right continuous composite web 130 and left continuous composite web 140, this nesting pattern may be suitable to form the ear portions of articles that are being formed in a front:front/back:back configuration. As shown, there are two front ear portions 180 on one side of the composite web 120 and there are two back ear portions 190 opposite the front ear portions 180. Once the composite web 120 is split for application to the absorbent article web 160, the right continuous composite web 130 may be sped up or slowed down in relation to the left continuous composite web 140 in order for sets of front ear portions 180 to be applied to adjacent front waist regions 185. Likewise, the relative speed of the right continuous composite web 130 and the left continuous composite web 140 should be timed so that sets of back ear portions 190 are applied to adjacent back waist regions 195.

In addition to depicting an alternative nesting pattern for the ear portions, FIG. 4 also depicts a composite web 120 that may include multiple materials for forming the ear portions. The composite web 120 includes a base web 100. The base web 100 material may be selected from a variety of materials but in one particular embodiment comprises a spunbond-meltblown-spunbond nonwoven material (“SMS” material). The base web 100 has a first outer surface 122 and second outer surface 124, opposed first longitudinal side edge 126 and second longitudinal side edge 128, and a longitudinal centerline 129 (representatively illustrated with a dashed line in FIG. 4). The base web 100 has a first longitudinally extending zone 132 lying between the longitudinal centerline 129 and the first longitudinal side edge 126. The base web 100 has a second longitudinally extending zone 134 lying between the longitudinal centerline 129 and the second longitudinal side edge 128. The first fastening material 108 is situated in the first longitudinally extending zone 132 on the first outer surface 122. The second fastening material 109 is situated in the second longitudinally extending zone 134 on the first outer surface 122. The first fastening material 108 and/or the second fastening material 109 may include a mechanical fastening hook material or other material suitable for mechanical fastening. By selectively situating the first fastening material 108 as a discrete piece in the first longitudinally extending zone 132 only the front ear portions 180 will include the first fastening material 108. By selectively situating the second fastening material 109 as a discrete piece in the second longitudinally extending zone 134 only the front ear portions 180 will include the second fastening material 109. The length or height of the front ear portions 180 is selected so that it is short enough to not include the third fastening material 110.

The composite web 120 may also include a third fastening material 110 that is applied in such a way that it runs down the center of the composite web 120 on the second outer surface 124. The third fastening material 110 may include a mechanical fastening hook material or other material suitable for mechanical fastening. By running the third fastening material 110 down the center of the composite web 120, only the ends of the back ear portions 190 will include the third fastening material 110. The composite web 120 may further include a fourth material 125 that forms part of the back ear portions 190 and part of the front ear portions 180. The fourth material 125 may be situated on the first outer surface 122 or the second outer surface 124. Exemplary fourth materials may include a stretchable or extensible material or a super-soft material.

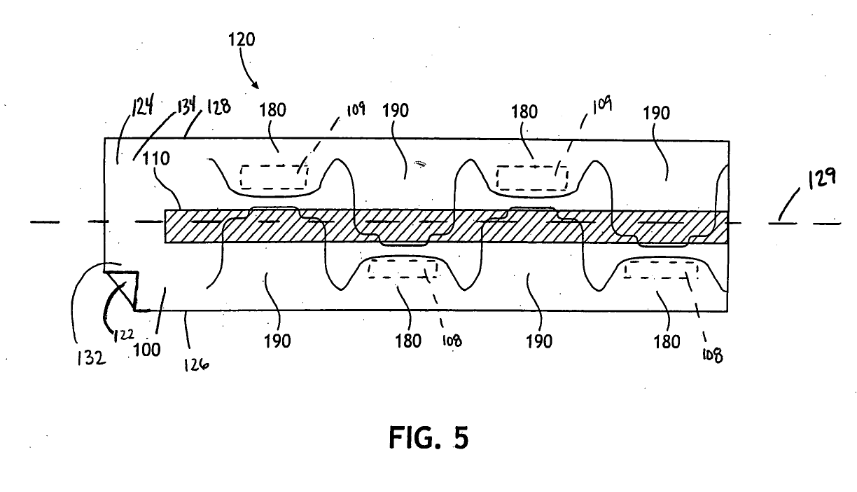

FIG. 5 representatively illustrates a composite web 120 that can be die-cut into front ear portions 180 and back ear portions 190. The shapes of the front ear portions 180 and the back ear portions 190 are selected so that the shapes can be “nested” with each other on the composite web 120. The nesting pattern depicted in FIG. 5 is similar to the pattern described in relation to FIG. 2. One difference is that the front ear portion 180 shape depicted in FIG. 5 includes a curved profile whereas the front ear portion 180 shape depicted in FIG. 2 includes a straight profile. The profiles of both the front ear portions 180 and the back ear portions 190 can be designed to have both curved and straight portions so long as the profiles can co-exist opposite each other when formed from the composite web 120. The nesting pattern of FIG. 5 is an example of how using the composite webs 120 of the invention can significantly reduce wasted materials; the front ear portions 180 and back ear portions 190 are closely nested together in such a way that there is minimal material remaining between them.

The composite web 120 depicted in FIG. 5 has a base web 100. The base web 100 has a first outer surface 122 and a second outer surface 124, an opposed first longitudinal side edge 126 and a second longitudinal side edge 128, and a longitudinal centerline 129 (representatively illustrated with a dashed line). The base web 100 has a first longitudinally extending zone 132 lying between the longitudinal centerline 129 and the first longitudinal side edge 126. The base web 100 has a second longitudinally extending zone 134 lying between the longitudinal centerline 129 and the second longitudinal side edge 128. The composite web 120 depicted in FIG. 5 includes a base web 100 material, a first fastening material 108, a second fastening material 109, and a third fastening material 110. The first fastening material 108 is situated in the first longitudinally extending zone 132 on the first outer surface 122. The second fastening material 109 is situated in the second longitudinally extending zone 134 on the first outer surface 122. The third fastening material 110 is situated on the second outer surface 124 and at least a portion of the third fastening material 110 is situated on the longitudinal centerline 129. The first, second, or third fastening materials 108, 109, and 110 may be a fastening material that can be attached to, or formed integrally with, the base web 100. The fastening materials can be formed of a soft, flexible polymer such as KRATON polymer, polyethylene, ethylene vinyl acetate, and metallocene film (adhesive-based) that can be printed or otherwise directly applied onto the base web 100.

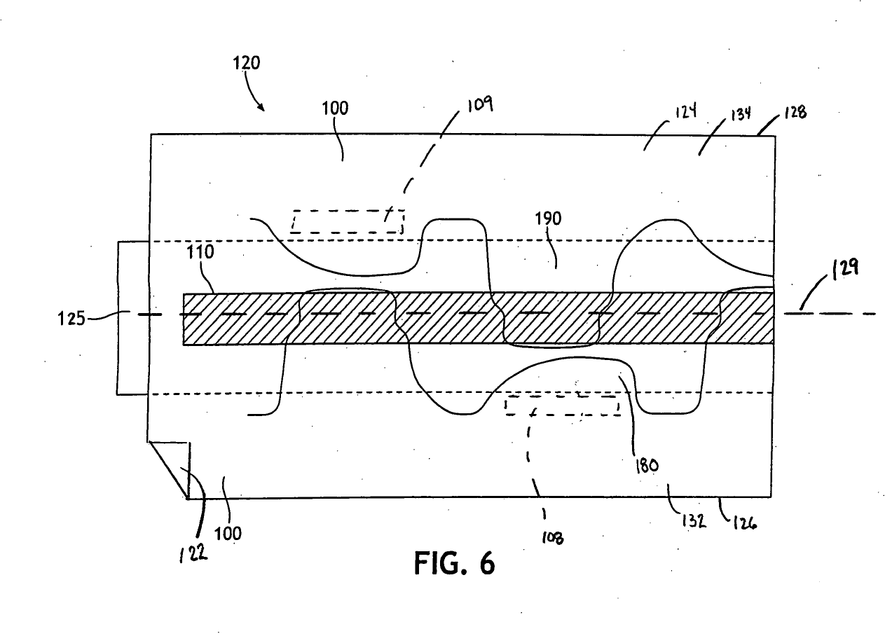

Another embodiment of front ear portions 180 and back ear portions 190 formed from the same material web is depicted in FIG. 6. FIG. 6 illustrates alternative shapes for both the front ear portions 180 and the back ear portions 190. These shapes illustrate that a variety of shapes may be selected and are still capable of being nested with each other on a common composite web 120. The use of multiple materials within one composite web 120 is also illustrated. For example, the composite web 120 may include a base web 100, a first fastening material 108, a second fastening material 109, a third fastening material 110, and a fourth material 125. The base web 100 has a first outer surface 122 and second outer surface 124, opposed first longitudinal side edge 126 and second longitudinal side edge 128, and a longitudinal centerline 129 (representatively illustrated with a dashed line in FIG. 6). The base web 100 has a first longitudinally extending zone 132 lying between the longitudinal centerline 129 and the first longitudinal side edge 126. The base web 100 has a second longitudinally extending zone 134 lying between the longitudinal centerline 129 and the second longitudinal side edge 128. The first fastening material 108 is situated in the first longitudinally extending zone 132 on the first outer surface 122. The second fastening material 109 is situated in the second longitudinally extending zone 134 on the first outer surface 122. The base web 100 may form the parts of the front ear portions 180 and the back ear portions 190 that will become the attachment areas of an absorbent article web 160, as illustrated in FIG. 1B. A first, second, and third fastening materials 108, 109, and 110 may be attached to or formed integrally with the base web 100. The third fastening material 110 may form the parts of the back ear portions 190 that will become a primary fastening surface for engagement with another fastening surface when used on an absorbent article. The first and second fastening materials 108 and 109 may form the parts of the front ear portions 180 that will become a secondary fastening surface for engagement with another fastening surface when used on an absorbent article. The first and second fastening materials 108 and 109 may be located on a first outer surface 122 of the base web 100. The third fastening material 110 may be located on a first outer surface 122 of the base web 100, a second outer surface 124 of the base web 100, or on both sides/surfaces of the base web 100. A fourth material 125 may likewise be attached to or integrally formed with the base web 100. The fourth material 125 may be the same type of material as the third fastening material 110 or it may be different. The fourth material 125 may form a portion of the back ear portions 190 and may form a portion of the front ear portions 180. The fourth material 125 may be an extensible or elastic material such as a NBL or SBL. Both the third fastening material 110 and the fourth material 125 may be located on one or more areas of the base web 100. In another aspect, the third fastening material 110 may be attached to or formed integrally with the fourth material 125.

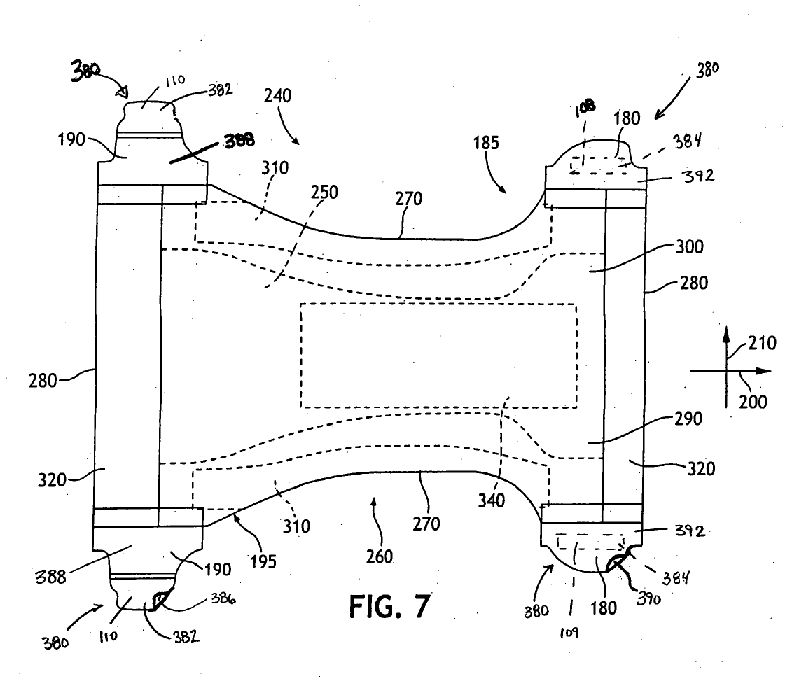

FIG. 7 representatively illustrates an example of a disposable absorbent article to which the front ear portions 180 and the back ear portions 190 formed from the same composite web 120, as previously discussed, may be attached. The absorbent article, which may be in the form of a disposable diaper, is generally indicated at 240. The absorbent article 240 is generally illustrated with the body facing surface towards the viewer. As illustrated in FIG. 7, the diaper 240 defines an absorbent 250, a front waist region 185, a back waist region 195, a crotch region 260 that extends between and connects the front and back waist regions 185 and 195, a longitudinal direction 200 and a lateral direction 210. The front waist region 185 includes the portion of the diaper 240 that, when worn, is positioned on the front of the wearer while the back waist region 195 includes the portion of the diaper 240 that, when worn, is positioned on the back of the wearer. The crotch region 260 of the diaper 240 includes the portion of the diaper 240 that, when worn, is positioned between the legs of the wearer and covers the lower torso of the wearer.

The diaper 240 defines a pair of laterally opposed side edges 270, a pair of longitudinally opposed waist edges 280, an interior surface 290 (or body facing surface) that is configured to contact the wearer, and an exterior surface (not shown) opposite the interior surface 290 that is configured to contact the wearer's clothing in use (or garment facing surface). The illustrated diaper 240 also includes an outer cover (not visible in FIG. 7) and a bodyside liner 300 that is connected to the outer cover in a superposed relation. An absorbent 250 is located between the outer cover and the bodyside liner 300. The laterally opposed side edges 270 of the diaper 240 are generally defined by the side edges of the outer cover that further define leg openings that may be curvilinear. The waist edges 280 of the diaper 240 are generally defined by the waist edges of the outer cover and define a waist opening that is configured to encircle the waist of the wearer when worn. The absorbent 250 is configured to contain and/or absorb any body exudates discharged from the wearer. The diaper 240 may further include leg elastics 310, containment flaps (not shown) and waist elastics 320 as are known to those skilled in the art. The diaper 240 may include a dual fastening system 380 that includes primary fasteners 382 that are part of the back ear portions 190 and secondary fasteners 384 that are part of the front ear portions 180. Each back ear portion 190 extends laterally outward from a side edge 270 in the back waist region 195, each back ear portion 190 has a garment facing first outer surface 386, a body facing second outer surface 388 and a third fastening material 110. The third fastening material 110 is situated on at least a portion of the second outer surface 388 of each back ear portion 190, the third fastening material 110 being configured to engage at least a portion of the garment facing surface of the outer cover and function as a primary fastener 382 for securing the diaper 240 about the waist of the wearer. Each front ear portion 180 extends laterally outward from a side edge 270 in the front waist region 185. Each front ear portion 180 has a garment facing first outer surface 390 and a body facing second outer surface 392. Each front ear portion 180 has at least one of a first fastening material 108 or a second fastening material 109, the first or second fastening material 108 or 109 being situated on at least a portion of the first outer surface 390 of each front ear portion 180, the first or second fastening material 108 or 109 being configured to engage at least a portion of the body facing surface of the liner 300 and function as a secondary fastener 384 for securing the diaper 240 about the waist of the wearer. It should be recognized that individual components of the diaper 240 may be optional depending upon the intended use of the diaper 240.

As illustrated in FIG. 7, the front and back ear portions 180 and 190 include curved edges and are in precise registration with the waist edges 280 of the diaper 240. More specifically, the front ear portions 180 are aligned with the waist edge 280 in the front waist region 185 of the diaper 240 and the back ear portions 190 are aligned with the waist edge 280 in the back waist region 195 of the diaper 240. This alignment or registration can assist with improved fit and performance of the diaper 240. For example, when a diaper 240 such as the one illustrated in FIG. 7 is applied to a baby, the caregiver may pull (apply tension) the back ear portions 190 around to the front waist region 185 and secure the third fastening material 110, functioning as a primary fasteners 382 to an exterior surface of the front waist region 185. Simultaneously, if the waist elastic 320 in the back waist region 195 is stretchable, the waist elastic 320 will be better tensioned and the diaper 240 will fit the baby better around the waist. Additionally, the first and/or second fastening material 108 or 109, functioning as secondary fasteners 384, will engage with an interior surface of the back waist region 195 to provide additional stability in the region wherein the front waist region 185 and back waist region 195 overlap. The secondary fasteners 384 work to reduce twisting which helps maintain proper fit.

The diaper 240 may be of various suitable shapes. For example, in the unfastened configuration as illustrated in FIG. 7, the diaper may have an overall rectangular shape, T-shape or an approximately hourglass shape. In the shown embodiment, the diaper 240 has a generally I-shape in an unfastened configuration. Examples of diaper configurations suitable for use in connection with the instant application and other diaper components suitable for use on diapers are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996, to Hanson et al., the disclosures of which are herein incorporated by reference where not contradictory.

The various components of the diaper 240 are integrally assembled together employing various types of suitable attachment means, such as adhesive, sonic bonds, thermal bonds or the like, as well as combinations thereof. In the shown embodiment, for example, the outer cover and bodyside liner 300 are assembled to each other and to the absorbent 250 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Alternatively, the absorbent 250 may be connected to the outer cover using conventional fasteners such as buttons, hook and loop type fasteners, adhesive tape fasteners, and the like. The other components of the diaper 240 may be suitably connected together using similar means. Similarly, other diaper components, such as the elastic members 320, may be assembled into the diaper 240 article by employing the above-identified attachment mechanisms. Desirably, the majority of the diaper components are assembled together using ultrasonic bonding techniques for reduced manufacturing cost.

The outer cover generally presents a garment facing surface of the diaper 240. More specifically, the outer cover has a body facing surface and a garment facing surface. The body facing surface of the outer cover is superposed with the liner 300. The outer cover may suitably be composed of a material which is either liquid permeable or liquid impermeable. It is generally preferred that the outer cover be formed from a material that is substantially impermeable to liquids. A typical outer cover can be manufactured from a thin plastic film or other flexible liquid-impermeable material. For example, the outer cover may be formed from a polyethylene film having a thickness of from about 0.013 millimeter (0.5 mil) to about 0.051 millimeter (2.0 mils). If it is desired to present the outer cover with a more clothlike feeling, the outer cover may be formed from a polyolefin film having a nonwoven web laminated to the exterior surface thereof, such as a spunbond web of polyolefin fibers. For example, a stretch-thinned polypropylene film having a thickness of about 0.015 millimeter (0.6 mil) may have thermally laminated thereto a spunbond web of polypropylene fibers. The polypropylene fibers have a thickness of about 1.5 to 2.5 denier per filament, which nonwoven web has a basis weight of about 17 grams per square meter (0.5 ounce per square yard). The outer cover may otherwise include bicomponent fibers such as polyethylene/polypropylene bicomponent fibers. Methods of forming such clothlike outer covers are known to those skilled in the art. The outer cover may also be an extensible outer cover such as the outer covers described in U.S. Pat. No. 6,552,245 issued Apr. 22, 2003 to Roessler et al., the entirety of which is incorporated herein where not contradictory.

Further, the outer cover may be formed of a woven or nonwoven fibrous web layer which has been totally or partially constructed or treated to impart a desired level of liquid impermeability to selected regions that are adjacent or proximate the absorbent 250. Still further, the outer cover may optionally be composed of a micro-porous “breathable” material which permits vapors to escape from the absorbent 250 while still preventing liquid exudates from passing through the outer cover. For example, the outer cover may include a vapor permeable non-woven facing layer laminated to a micro-porous film. Suitable “breathable” outer cover materials are described in U.S. Pat. No. 5,695,868 issued to McCormack et al. and U.S. Pat. No. 5,843,056 issued Dec. 1, 1998 to Good et al., the descriptions of which are hereby incorporated by reference where not contradictory. Still further, the outer cover may also be an elastomeric material such as a stretch-thermal laminate (STL), neck-bonded laminate (NBL), or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference where not contradictory. The outer cover can also be embossed or otherwise provided with a matte finish to provide a more aesthetically pleasing appearance.

The bodyside liner 300, as representatively illustrated in FIG. 7, suitably presents a bodyfacing surface of the diaper 240 that is compliant, soft feeling, and nonirritating to the wearer's skin. Further, the bodyside liner 300 may be less hydrophilic than the absorbent 250, to present a relatively dry surface to the wearer, and may be sufficiently porous to be liquid permeable, permitting liquid to readily penetrate through its thickness. The liner 300 has a body facing surface and a garment facing surface. The garment facing surface of the liner is superposed with the body facing surface of the outercover. A suitable bodyside liner 300 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 300 is suitably employed to help isolate the wearer's skin from liquids held in the absorbent 250. The bodyside liner 300 can also be made from extensible materials as are described in U.S. Pat. No. 6,552,245 that issued to Roessler et al. on Apr. 22, 2003, the entirety of which is incorporated herein by reference where not contradictory.

Various woven and nonwoven fabrics can be used for the bodyside liner 300. For example, the bodyside liner may be composed of a meltblown or spunbond web of polyolefin fibers. The bodyside liner 300 may also be a bonded-carded web composed of natural and/or synthetic fibers. The bodyside liner 300 may be composed of a substantially hydrophobic material, and the hydrophobic material may optionally be treated with a surfactant or otherwise processed to impart a desired level of wettability and hydrophilicity. In a particular embodiment of the present invention, the bodyside liner 300 is made from a nonwoven, spunbond, polypropylene fabric composed of about 1.5-2.5 denier fibers formed into a web having a basis weight of about 20 grams per square meter and a density of about 0.13 grams per cubic centimeter. The fabric may be surface treated with about 0.3 weight percent of a surfactant commercially available from Hodgson Textile Chemicals, Inc. under the trade designation AHCOVEL Base N-62. The surfactant may be applied by any conventional means, such as spraying, printing, brush coating or similar techniques. The surfactant may be applied to the entire bodyside liner 300 or may be selectively applied to particular sections of the bodyside liner 300, such as the medial section along the longitudinal centerline of the diaper, to provide greater wettability of such sections. The bodyside liner 300 may further include a lotion or treatment applied thereto that is configured to be transferred to the wearer's skin. Suitable compositions for application to the bodyside liner 300 are described in U.S. Pat. No. 6,149,934 that issued to Krzysik et al. on Nov. 21, 2000, the entirety of which is incorporate herein by reference where not contradictory.

The absorbent 250 of the diaper 240, as representatively illustrated in FIG. 7, may suitably be composed of a matrix of hydrophilic fibers, such as a web of cellulosic fluff, mixed with particles of a high-absorbency material commonly known as superabsorbent material. In a particular embodiment, the absorbent 250 includes a matrix of cellulosic fluff such as wood pulp fluff and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent 250 to better contain and absorb body exudates. The concentration of the superabsorbent particles may also vary through the thickness of the absorbent 250. Alternatively, the absorbent 250 may include a laminate of fibrous webs and superabsorbent material or other suitable means of maintaining a superabsorbent material in a localized area.

The absorbent 250 may have any of a number of shapes. For example, the absorbent may be rectangular, I-shaped, or T-shaped. It is generally preferred that the absorbent 250 be narrow in the crotch region 260 of the diaper 240. It has been found that the absorbent 250 of the present invention is particularly useful when the width dimension in the crotch region 260 is from about 2.5 to about 12.7 centimeters (1.0 to about 5.0 inches), desirably no more than about 7.6 centimeters (3.0 inches) and more desirably no more than about 5.1 centimeters (2.0 inches). The narrow crotch width dimension of the absorbent 250 allows the absorbent 250 to better fit between the legs of the wearer. The size and the absorbent capacity of the absorbent 250 should be compatible with the size of the intended wearer and the liquid loading imparted by the intended use of the absorbent article.

The high-absorbency material can be selected from natural, synthetic, and modified natural polymers and materials. The high-absorbency materials can be inorganic materials, such as silica gels, or organic compounds, such as crosslinked polymers. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va. and DOW DRYTECH 2035LD polymer available from Dow Chemical Co. located in Midland, Mich. As a general rule, the high absorbency material is present in the absorbent body in an amount of from about 5 to about 90 weight percent based on total weight of the absorbent 250.

Optionally, a substantially hydrophilic tissue wrapsheet may be employed to help maintain the integrity of the airlaid fibrous structure of the absorbent 250. The tissue wrapsheet is typically placed about the absorbent body over at least the two major facing surfaces thereof and composed of an absorbent cellulosic material, such as creped wadding or a high wet-strength tissue. In one aspect of the invention, the tissue wrapsheet can be configured to provide a wicking layer that helps to rapidly distribute liquid over the mass of absorbent fibers comprising the absorbent body. The wrapsheet material on one side of the absorbent fibrous mass may be bonded to the wrapsheet located on the opposite side of the fibrous mass to effectively entrap the absorbent 250. There may also be a surge layer 340 located between the absorbent body 250 and the liner 300 to facilitate the distribution of fluid during intake.

The disposable diaper 240 may include a pair of containment flaps (not shown in FIG. 7) that are configured to provide a barrier to the lateral flow of body exudates. The containment flaps may be located along the laterally opposed side edges 270 of the diaper adjacent the side edges of the absorbent 250. Such containment flaps are generally well known to those skilled in the art. For example, suitable constructions and arrangements for containment flaps are described in U.S. Pat. No. 4,704,116 issued Nov. 3, 1987, to K. Enloe, the disclosure of which is hereby incorporated by reference where not contradictory.

The diaper 240 of the different aspects of the present invention may further include elastics at the waist edges 280 and side edges 270 of the diaper 240 to further prevent leakage of body exudates and support the absorbent 250. For example, the diaper 240 of the present invention may include a pair of leg elastic members 310 that are connected to the laterally opposed side edges 270 of the diaper 240 in the crotch region 260. The diaper 240 may also include a pair of waist elastic members 320 that is connected to the longitudinally opposed waist edges 280 of the diaper 240. The leg elastics 310 and waist elastics 320 are generally adapted to fit about the legs and waist of a wearer in use to maintain a positive, contacting relationship with the wearer to effectively reduce or eliminate the leakage of body exudates from the diaper 240.

Materials suitable for use as the leg elastics 310 and waist elastics 320 are well known to those skilled in the art. Exemplary of such materials are sheets or strands or ribbons of a polymeric, elastomeric material that are adhered to the outer cover in a stretched position, or that are attached to the outer cover while the outer cover is pleated, such that elastic constrictive forces are imparted to the outer cover. The leg elastics 310 may also include such materials as polyurethane, synthetic and natural rubber.

The diaper 240 of the different aspects of the present invention may further include a fit panel (not illustrated in FIG. 7) superimposed adjacent to the waist edge 280 in at least one of the waist sections 185 and 195, to provide a more comfortable, contouring fit about the wearer. For example, the diaper 240 may include a fit panel superimposed adjacent the waist edge 280 on either the interior or exterior surface of the diaper 240. Or there may be a fit panel located on both surfaces of the diaper 240 simultaneously. The diaper may include a fit panel disposed in both waist sections 185 and 195 and desirably the diaper includes a fit panel in at least the rear waist section 195. Desirably, the fit panel is extensible or elastomeric. Absorbent articles including such a fit panel and methods of making the same are further described in U.S. Pat. No. 6,336,922 issued Jan. 8, 2002 to VanGompel et al., the entirety of which is hereby incorporated by reference where not contradictory.

As previously described, in various aspects, the diaper 240 can further include a dual fastening system 380 for securing the diaper 240 about the waist of the wearer. In some embodiments, the dual fastening system 380 can include a pair of primary fasteners 382 and a pair of secondary fasteners 384 that may be used for joining the front and back waist sections 185 and 195 to each other during application of the diaper 240 to a wearer. An attachment panel (not illustrated in FIG. 7) may be located on the outer cover to which the primary fasteners 382 may be releasably engaged. The disposable diaper 240 of the present invention may include an attachment panel located on the outer cover in one of the waist regions 185 and 195 on the exterior surface of the diaper 240. In such a configuration, the primary fastener 382 may be refastenably engaged with the attachment panel to maintain the diaper 240 about the waist of the wearer. The attachment panel may include two separate panels located along the opposed side edges of the diaper 240 in one of the waist regions 185 and 195 of the diaper 240. Alternatively, the attachment panel may include a single piece of material that extends substantially across the respective waist region of the diaper 240. Alternatively, the primary fasteners 382 may be releasably engageable directly with the garment facing surface of the outer cover.

The dual fastening system 380 of the present invention further includes a pair of secondary fasteners 384 to provide improved securement of the diaper 240 about the waist of the wearer. For example, as representatively illustrated in FIG. 7, the diaper 240 may include a pair of secondary fasteners 384 situated on the front ear portions 180 in the front waist region 185. The secondary fasteners 384 are configured to encircle the hips of the wearer and engage the body facing surface of the liner 300 in the back waist region 195 of the diaper 240. Suitably, the secondary fasteners 384 are hook type fasteners which are releasably engageable directly with the body facing surface of the liner 300. Alternatively, the diaper 240 may include one or more fastening panels (not illustrated) on the body facing surface of the back waist region 195 to which the secondary fasteners 384 are releasably engageable.

Suitable fastening materials to provide the engageable portions of the primary fasteners 382 and/or the secondary fasteners 384 are well known to those skilled in the art and can include adhesive tape tab fasteners, hook and loop fasteners, mushroom fasteners, snaps, pins, belts and the like, and combinations thereof. The primary fasteners 382 may include hook type fastening materials and the outer cover may be configured to function as a complementary loop type fastener. The secondary fasteners 384 may include hook type fastening materials and the liner may be configured to function as a secondary loop type fastener. Alternatively, an attachment panel may be provided on the diaper 240 on either the outer cover, liner, or both to function as a complementary loop type fastener. Desirably, the primary fasteners 382 may include hook type fasteners that are releasably engageable directly with the outer cover of the diaper 240. Desirably, the secondary fasteners 384 include hook type fasteners that are releasably engageable directly with the liner 300 of the diaper 240. Such an arrangement provides the ability to vary the size of the waist opening in very small increments over a wide range to fit the waist of the wearer. Particular examples of hook fastening materials can include VELCRO HTH 858 or VELCRO HTH 823, or a similar hook material available from Velcro Industries B.V., Amsterdam, Netherlands or affiliates thereof. The primary fasteners 382 and/or the secondary fasteners 384 may also include adhesive-type materials known in the art and having the desired strength for joining the waist regions 185 and 195 together when the diaper 240 is applied. Further, the primary fasteners 382 and/or the secondary fasteners 384 may include materials that are capable of cohesively attaching to or engaging an opposite portion of the diaper 240 for fastening about the wearer.

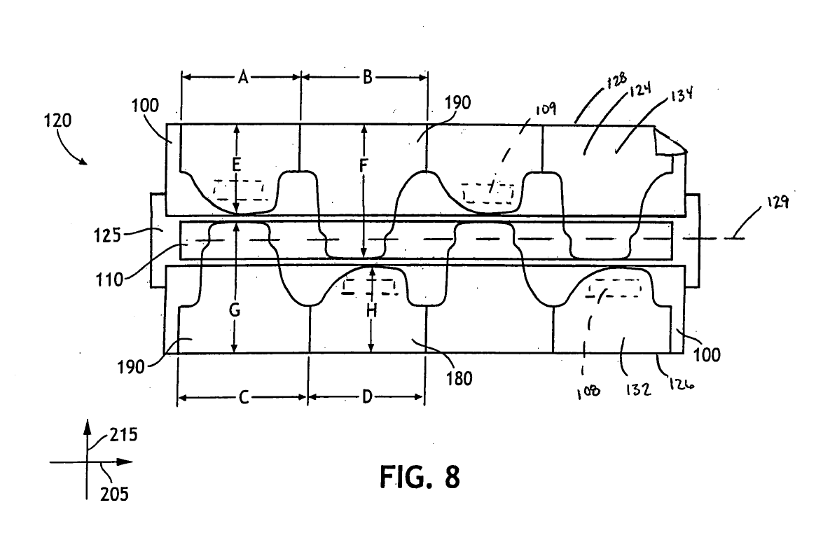

As previously described herein, the primary fastener 382 portion of the diaper 240 may be incorporated into the back ear portion 190 formed from a composite web 120 together with the secondary fastener 384 incorporated into the front ear portion 180. FIG. 8 representatively illustrates an example of dimensions of front and back ear portions 180 and 190 that may be nested together and therefore formed from the same material web 120. The composite material web 120 depicted in FIG. 8 includes a base web 100, a first fastening material 108, a second fastening material 109, a third fastening material 110 and a fourth material 125. The material web 120 has a machine direction 205 and a cross direction 215. The length of the front and back ear portions 180 and 190 is aligned with the cross direction 215 of the material web 120 and the width of the front and back ear portions 180 and 190 is aligned with the machine direction 205 of the material web 120. As representatively illustrated in FIG. 8, the fourth material 125 is underneath the base web 100 and the third fastening material 110. The front ear portions 180 include the base web 100, one of the first fastening material 108 or the second fastening material 109, and a portion of the fourth material 125. The front ear portions 180 do not, in this example, include the third fastening material 110. The back ear portions 190 include the base web 100, the third fastening material 110 and the fourth material 125. The base web 100 material is provided in two sections in this example; both sections are overlying the fourth material 125. In this example, both sections of the base web 100 have a length in the cross direction 215 of the material web 120 of 55 mm. The third fastening material 110 has a length in the cross direction 215 of the material web 120 of 23 mm. The fourth material 125 has a length in the cross direction 215 of the material web 120 of 56 mm.

Two diagonally opposed front ear portions 180 shown in FIG. 8 have widths represented by letters “A” and “D”. In one particular embodiment, the widths of the front ear portions 180 are approximately 78 mm and “A” and “D” are approximately equal to each other. Two diagonally opposed back ear portions 190 shown in FIG. 8 have widths represented by letters “B” and “C”. In this embodiment, the widths of the back ear portions 190 are approximately 86 mm and “B” and “C” are approximately equal to each other. The combined width of a pair of adjacent front and back ear portions 180 and 190 is represented by “A+B” and “C+D” is approximately equal to 164 mm. Further, the sum “A+B” is approximately equal to the sum “C+D”. The two diagonally opposed front ear portions 180 have lengths represented by letters “E” and “H”. The lengths of the front ear portions 180 are approximately 55 mm, approximately the same as the lengths of the base web 100 material in the cross direction 215. The two diagonally opposed back ear portions 190 have lengths represented by letters “F” and “G”. The lengths of the back ear portions 190 are approximately 78 mm, approximately the same as the sum of the lengths of one of the base webs 100 and the length of third fastening material 110 plus the approximate gap between webs 100 and 110 in the cross direction 215. The lengths of the back ear portions 190 depicted in FIG. 8 also include a section of the fourth material 125. The lengths of the back ear portions 190 are generally greater than the lengths of the front ear portions 180; that is, 78 mm is greater than 55 mm. Additionally, the sum of the lengths of the back ear portions (“F+G”) is generally greater than the sum of the lengths of the front ear portions (“E+H”); that is, approximately 156 mm is greater than 110 mm. With the example of a nested ear pattern of the invention shown in FIG. 8, it may be desirable for the fourth material 125 to be a mechanical hook material that is capable of serving as a fastening material capable of engagement with another portion of an absorbent article to which the front and back ear portions 180 and 190 are eventually attached.

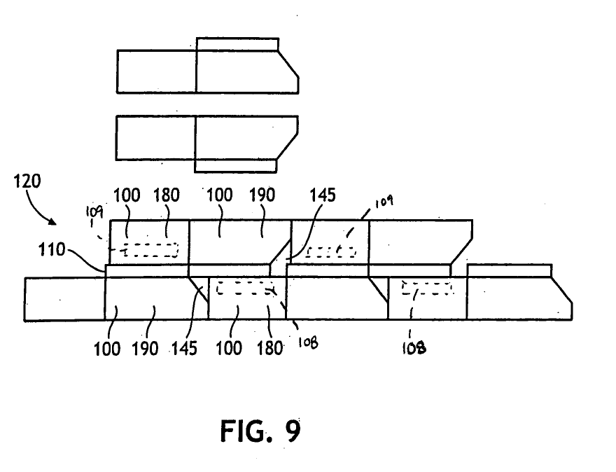

The present invention may also be utilized for the formation of prefastened absorbent articles. Prefastened articles may be pant-like in configuration so that the wearer puts their legs through leg openings and pulls the article up and over their hips. FIG. 9 representatively shows a material web 120 from which back ear portions 190 and front ear portions 180 may be formed for use on a prefastened absorbent article. The back ear portions 190 and front ear portions 180 may be more rectangular in shape and their nesting pattern may be such as to result in minimal trim waste 145. After the back ear portions 190 and front ear portions 180 are applied to an absorbent chassis, the back ear portions 190 may be attached to the front ear portions 180 in order to provide the “prefastened” configuration of the article. The back ear portions 190 and the front ear portions 180 may be attached by techniques know to those of skill in the art such as adhesive, thermal and ultrasonic bonding. Additionally, a first fastening material 108, a second fastening material 109, and a third fastening material 110 may be included in the composite material web 120. The first, second and third fastening materials 108, 109, and 110 are fastening materials capable of engaging and attaching to the base web material 100 used to form the other portions of the front and back ear portions 180 and 190. For example, the first, second, or third fastening materials 108, 109, 110 may be a mechanical hook material. As depicted in FIG. 9, the shapes of the front and back ear portions 180 and 190 may be selected such that only the back ear portions 190 include the third fastening material 110 and only the front ear portions 180 include one of the first fastening material 108 or the second fastening material 109.

While the invention has been described in detail with respect to specific aspects thereof, it will be appreciated that those skilled in the art, upon attaining an understanding of the foregoing, may readily conceive of alterations to, variations of and equivalents to these aspects. Accordingly, the scope of the present invention should be assessed as that of the appended claims and any equivalents thereto.

1. A composite web comprising a base web having opposed first and second outer surfaces, a longitudinal centerline, opposed first and second longitudinal side edges, a first longitudinally extending zone lying between the longitudinal centerline and the first longitudinal side edge, a second longitudinally extending zone lying between the longitudinal centerline and the second longitudinal side edge, and first, second and third fastening materials, the first fastening material being situated in the first longitudinally extending zone on the first outer surface, the second fastening material being situated in the second longitudinally extending zone on the first outer surface, and at least a portion of the third fastening material being situated on the longitudinal centerline on the second outer surface.

2. The composite web of claim 1, wherein the first fastening material is a discontinuous strip of material and the second fastening material is a discontinuous strip of material.

3. The composite web of claim 2, wherein the first fastening material and the second fastening material are configured in offset relation with one another.

4. The composite web of claim 3, wherein at least a portion of the first fastening material is situated adjacent a longitudinal side edge.

5. The composite web of claim 4, wherein at least a portion of the second fastening material is situated adjacent a longitudinal side edge.

6. The composite web of claim 5, wherein the first fastening material is the same as the second fastening material.

7. The composite web of claim 6, wherein the third fastening material is a discontinuous strip of material.

8. The composite web of claim 1, wherein at least a portion of the first fastening material is situated adjacent a longitudinal side edge.

9. The composite web of claim 8, wherein at least a portion of the second fastening material is situated adjacent a longitudinal side edge.

10. The composite web of claim 9, wherein the first fastening material is the same as the second fastening material.

11. A disposable absorbent article having opposed side edges, a front waist region, a back waist region, a crotch region extending between and connecting the waist regions, a longitudinal direction and a lateral direction, the disposable absorbent article comprising:
an outer cover having a body facing surface and a garment facing surface;
a liner superposed over the body facing surface of the outer cover, the liner having a body facing surface and a garment facing surface;
an absorbent core disposed between the garment facing surface of the liner and the body facing surface of the outer cover; and
a dual fastening system, the dual fastening system comprising a pair of front ear portions and a pair of back ear portions;
the pair of front ear portions and the pair of back ear portions are formed from a composite web, the composite web comprising: a base web having opposed first and second outer surfaces, a longitudinal centerline, opposed first and second longitudinal side edges, a first longitudinally extending zone lying between the longitudinal centerline and the first longitudinal side edge, a second longitudinally extending zone lying between the longitudinal centerline and the second longitudinal side edge, and first, second, and third fastening materials, the first fastening material being situated in the first longitudinally extending zone on the first outer surface, the second fastening material being situated in the second longitudinally extending zone on the first outer surface, and at least a portion of the third fastening material being situated on the longitudinal centerline on the second outer surface;
each back ear portion extending laterally outward from a side edge in the back waist region, each back ear portion having a garment facing first outer surface, a body facing second outer surface and the third fastening material, the third fastening material being situated on at least a portion of the second outer surface of each back ear portion, the third fastening material being configured to engage at least a portion of the garment facing surface of the outer cover; and
each front ear portion extending laterally outward from a side edge in the front waist region and having a garment facing first outer surface, a body facing second outer surface and one of the first fastening material or the second fastening material, the first or second fastening material being situated on at least a portion of the first outer surface of each front ear portion, the first and second fastening materials being configured to engage at least a portion of the body facing surface of the liner.

12. The disposable absorbent article of claim 11, wherein:
the first fastening material is a discontinuous strip of material;
the second fastening material is a discontinuous strip of material; and
the first fastening material and the second fastening material are configured in offset relation with one another when part of the composite web.

13. The disposable absorbent article of claim 11, wherein the first fastening material is similar to the second fastening material.

14. The disposable absorbent article of claim 11, wherein the third fastening material is a discontinuous strip of material when part of the composite web.

15. The disposable absorbent article of claim 11, wherein
the first fastening material comprises discrete pieces being entirely located in the first longitudinally extending zone of the composite web; and
the second fastening material comprises discrete pieces being entirely located in the second longitudinally extending zone of the composite web.

16. The disposable absorbent article of claim 11, wherein the first fastening material, the second fastening material, and the third fastening material are hook type fastening materials and the outer cover is configured to function as a complementary loop type fastener and the liner is configured to function as a complementary loop type fastener.

17. The disposable absorbent article of claim 11, further comprising at least one attachment panel located on the garment facing surface of the outer cover to which the third fastening material may be releasably engaged.

18. A method of making a disposable absorbent article that defines a front waist region, a back waist region, a crotch region that extends between and connects the waist regions, a longitudinal direction and a lateral direction, the method comprising:
providing a first continuous web, the first continuous web comprising a base web having opposed first and second outer surfaces, opposed first and second longitudinal side edges, a longitudinal centerline, a first longitudinally extending zone lying between the longitudinal centerline and the first longitudinal side edge, a second longitudinally extending zone lying between the longitudinal centerline and the second longitudinal side edge, and first, second, and third fastening materials, the first fastening material being situated in the first longitudinally extending zone on the first outer surface, the second fastening material being situated in the second longitudinally extending zone on the first outer surface, and at least a portion of the third fastening material being situated on the longitudinal centerline on the second outer surface,
selectively cutting the first continuous web to form two ear portion webs wherein each ear portion web includes a repeating pattern of a back ear portion and a front ear portion, the back ear portion comprising the third fastening material, the front ear portion comprising one of the first fastening material or the second fastening material;
cutting and orienting the two ear portion webs to form pairs where each pair includes one of the back ear portions and one of the front ear portions;
providing a second continuous web of interconnected absorbent chassis wherein each of said absorbent chassis defines an exterior surface, an interior surface opposite the exterior surface, a pair of laterally opposed side edges and a pair of longitudinally opposed waist edges;
attaching the pairs of ear portions on both of the laterally opposed side edges of the interconnected absorbent chassis wherein the pairs of ear portions are attached in alignment with the waist edges of adjacent absorbent chassis; and
selectively cuffing the second continuous web of interconnected absorbent chassis into discrete absorbent articles wherein each discrete absorbent article includes two laterally opposed front ear portions and two laterally opposed back ear portions, wherein the two laterally opposed front ear portions comprise one of the first or second fastener material and the two laterally opposed back ear portions comprise the third fastener material.

19. The method of claim 18 wherein the cutting and orienting step includes crossing the two ear portion webs and selectively cutting the two ear portion webs.

20. The method of claim 18 wherein the cutting and orienting step includes cutting the two ear portion webs to form pairs and rotating each pair 180 degrees.
